(19) United States
(12) Patent Application Publication
(10) Pub. No.: US 2006/0241606 A1
(43) Pub. Date: Oct. 26, 2006

(54) Plate device

(75) Inventors: Oz Vachtenberg, Tel-Aviv (IL); Ronen Shavit, Tel-Aviv (IL); Mordechay Beyar, Caesarea (IL)

Correspondence Address: WOLF, BLOCK, SCHORR & SOLIS-COHEN LLP, 250 PARK AVENUE, NEW YORK, NY, 10177, US

(73) Assignee: Disc-o-Tech, Ltd., Herzila Pituach (IL)

(21) Appl. No.: 10/560,628

(22) PCT Filed: Jun. 13, 2004
(86) PCT No.: PCT/IL04/00508

§ 371 Date: May 30, 2006

Related U.S. Application Data
(60) Provisional application No. 60/477,784, filed on Jun. 12, 2003.

(51) Int. Cl. A61F 2/30

(52) U.S. Cl. 606/65

(57) ABSTRACT

A hip plate, comprising: a plate body adapted to be attached to a femur or other bone; and at least one lag screw adapted for insertion into a femoral head or other bone protrusion and rotationally lockable to said plate.

### RELATED PATENTS

This application claims the benefit under 119(e) of US provisional application 60/477,784. This application is related to PCT/IL03/00052, filed Jan. 22, 2003 and published as WO 03/061495. This application is also related to a PCT application PCT/IL00/00471, filed Aug. 3, 2000, published as WO 01/54598 A1. The disclosures of all of these applications are incorporated herein by reference.

### FIELD OF INVENTION

The present invention relates to hip bone support, for example, using minimally invasive techniques.

### BACKGROUND OF THE INVENTION

Hip plate-screw systems have become one of the treatments of choice for management of unstable and stable intertrochanteric-, pertrochanteric- and base-of-femoral-neck fractures. Such systems are generally composed of a plate and screw(s) inserted into the femoral head (“lag screw”), as well as screws locking the plate to the femoral shaft.

Implantation of hip plate-screw systems are usually performed using an open surgical approach, however, a less invasive approach has been described (e.g., U.S. Pat. Nos. 4,465,065 and 5,429,641, the disclosures of which are incorporated herein by reference). The plate of a device implanted in an open approach typically has attached to the plate, an angled integral barrel for lag screw guiding, and distal holes formed in the plate for screws intended to lock the plate to the bone. In the less invasive approach the plate and barrel (which is connected to the lag screw) are provided as separate components, and assembled during surgery after the plate is positioned on the lateral cortex of the femoral bone. However, the rotational stability is compromised if only one lag screw in used. The barrel (with the lag screw) is connected to the plate intra-operatively, within the patient body, by screwing it into a threaded hole in the plate. In order to achieve rotational stability of the fractured bone, a second lag screw is introduced, an action that might lead to difficulties in performing the surgical procedure and might result in complications: having two lag screws within the femoral neck may damage the neck cortical bone and results, for example, in cut off.

### SUMMARY OF THE INVENTION

An aspect of some embodiments of the invention relates to a plate device, for example a hip plate-peg device in which a barrel guiding the peg is rotationally locked to the plate. In an exemplary embodiment of the invention, the device comprises a plate body, vertically placed and which contacts the external femoral lateral cortex; a plate barrel (“barrel”), for guiding a lag screw (“hip peg”), connected at an angle to plate body; a lag screw (“hip peg”), inserted through barrel into the femoral head; one or more cortical screws, that distally lock plate body to the bone; and a hip pin, optionally inserted in cases of comminuted fractures. In another example, the plate is attached to an upper arm bone and the lag screw is attached the head of the bone (e.g., humerus head), which fits in the shoulder socket.

In an exemplary embodiment of the invention, plate barrel is connected to plate body by a thread, and locked to it by rotating a bolt in the plate body, for example with a designated screwdriver, until bolt penetrates a matching slot in barrel. In another exemplary embodiment of the invention, barrel is connected to plate body by a spiral thread, and their locking is achieved automatically at the end of barrel rotational introduction/connection, by the insertion of a clamping snap in barrel into a designated slot in plate body.

In one embodiment of the invention, only one lag screw (“hip peg”) is inserted through the femoral neck into the femoral head. A potential advantage of inserting a single hip peg is reducing the possibility of damaging the femoral neck bone and complications thereof. Optionally, in cases of unstable/comminuted fractures, a locking hip pin may be inserted as well.

In one embodiment of the invention, hip peg has an expandable distal portion, which expands within the femoral head following hip peg insertion.

In an exemplary embodiment of the invention, relative rotation between the peg and the plate is prevented. In one example, the peg has an oval or other non-circular cross-section. In an alternative embodiment, a protrusion with a matching slot are used to prevent rotation. Optionally a same protrusion as used for locking the barrel to the plate.

In an exemplary embodiment of the invention, expandable distal section of hip peg is constructed from an expanding, metal thin membrane with protrusions, which is folded to gain a reduced configuration. Upon expansion, said section is unfolded to gain its expanded configuration. In an exemplary embodiment of the invention, said protrusions of expandable distal section are constructed as longitudinal bars, for example three bars. A potential advantage of such expanded configuration and other similar expanded configurations is enhancement of hip peg abutment within the femoral head. Another potential advantage of hip peg expansion is compression of trabaecular bone following expansion, which may lead to a better fixation inside bone.

In an exemplary embodiment of the invention, at least part of expanded portion is metallurgical treated, to have better elongation properties.

In an exemplary embodiment of the invention, the hip peg is expanded by pressurized liquid, for example sterile saline, using a manual pump. In an exemplary embodiment of the invention, the hip peg includes a one-way valve, assuring pressurized liquid remains within hip peg distal section. Alternatively, the hip peg does not contain such a valve, and hip peg remains in its expanded configuration due to plastic deformation of the inflated area.

Optionally, the axial sliding motion potential of hip peg is limited. Potential advantages of such limited sliding are enhancement of callus formation and reduction the risk of bone shortening and limited joint movement as well as of hip peg migration into acetabulum.

In an exemplary embodiment of the invention, the sliding motion of hip peg is limited by rotating a bolt in plate body, until it penetrates, through plate barrel, a matching slot in hip peg shaft.

Optionally, such an axial motion limitation is used in other implantable sliding peg systems.

In an exemplary embodiment of the invention, axial motion of the peg is limited by the barrel and rotational motion of the peg is also limited by the barrel. Both axial and rotational motion of the barrel are limited its connection to the plate.

An aspect of some embodiments of the invention relates to a method of treating proximal femoral fractures by minimally invasive implantation of hip plate-screw device-, in which a plate body and a barrel are connected and locked to each other intra-operatively (within the patient body). Optionally, only a single hip peg is used. Such minimally invasive procedure enables exposure of the bone without performing a long and wide incision in the overlaying tissue and skin, while still providing high rotational stability with only one hip peg inserted into the femoral head.

Optionally, the plate is designed for dissecting tissue during insertion.

An aspect of some embodiments of the invention relates to a design for a hip plate which is more resistant to clogging by bone chips. In an exemplary embodiment of the invention, interlocking sections have channels wide enough to allow any stray bone chip to be pushed away. In one example, a wide and single tier threading is used, so bone chips cannot be caught between adjacent threads. In another example, a snap connection comprises a snap which is surrounded on three sides by a slot wide enough for passage of bone chips. Optionally, the snap is strong enough and strongly enough elastically urged, so that it can crush any interfering bone chips.

An aspect of some embodiments of the invention relates to a device for extraction of hip peg from the body, if required. In an exemplary embodiment of the invention, the device is hydraulically powered, for example, using a same pressure source as used for peg inflation. In an exemplary embodiment of the invention, the device engages a peg to be removed and pulls it backwards. Optionally, the device also releases a valve so that pressure in the peg is reduced. Optionally, the device includes a tube which collapses an expended tip of the peg.

In an exemplary embodiment of the invention, a hip peg removal device comprises: a rod, connected to hip peg; a cylinder containing a piston that is connected to said rod; and an over tube, connected to said cylinder, into which hip peg is pulled.

In an exemplary embodiment of the invention, hip peg and rod are connected to each other by a threading.

In an exemplary embodiment of the invention, the cylinder is sealed with o-rings. Optionally, 2 o-rings are used.

In an exemplary embodiment of the invention, the hip peg removal device is connected to a manual pump, which delivers pressurized liquid, for example sterile saline, into cylindrical chamber. Once sufficient pressure is built in the chamber, the piston moves backwards and thus pulls hip peg into tube.

In one embodiment of the invention, during extraction, the diameter of an expanded distal portion of the hip peg is reduced while passing through a tube having diameter smaller than the diameter of the hip peg expanded section. In an exemplary embodiment of the invention, reduction in diameter is achieved by piercing a one-way valve inside hip peg, for example while connecting the hip peg to removal device rod, to allow pressure decrease and easier removal. In another exemplary embodiment of the invention, the diameter of the hip peg expanded section is reduced by mechanical forces, exerted while the hip peg is pulled through a tube that has a smaller diameter than the diameter of the hip peg expanded portion.

There is thus provided in accordance with an exemplary embodiment of the invention, a hip plate, comprising:

a plate body adapted to be attached to a bone; and

at least one lag screw adapted for insertion through the plate and into a bone section that is offset from said bone and rotationally lockable to said plate.

In an exemplary embodiment of the invention, said bone is a femur and wherein said offset is a femoral head.

In an exemplary embodiment of the invention, the plate comprises a barrel guide having an inner diameter adapted to contain a shaft of said lag screw and axially guide a motion of said lag screw. Optionally, said barrel is rotationally locked to said plate and wherein said screw is rotationally locked to said barrel. Alternatively or additionally, said barrel guide is adapted for attachment to said plate after said plate is implanted in a human body. Alternatively or additionally, said barrel is attached to said plate using a threading. Alternatively or additionally, said barrel is axially locked to said plate preventing motion of said barrel along a main axis thereof.

In an exemplary embodiment of the invention, said barrel is locked using a manually positioned locking element. Alternatively or additionally, said barrel is locked using a self-engaging element.

In an exemplary embodiment of the invention, said lag screw has an expandable distal end. Optionally, said distal end is inflatable. Alternatively or additionally, said distal end comprises a plurality of protrusions. Optionally, said protrusions comprise axial bars.

In an exemplary embodiment of the invention, said expandable end is treated to increase elongation.

In an exemplary embodiment of the invention, said lag screw comprises a one way fluid valve. Optionally, said valve is adapted to release said fluid when said valve is axially depressed towards said a distal end of said lag screw.

In an exemplary embodiment of the invention, said lag screw includes an axial motion limiter. Optionally, said limiter comprises a slot adapted to be engaged by a matching protrusion. Alternatively or additionally, wherein said limiter allows some axial motion.

In an exemplary embodiment of the invention, a shaft section of said peg has a cross-section that is not circular. Optionally, said peg has an oval cross-section.

In an exemplary embodiment of the invention, an end of said plate is sharp enough to push away tissue.

In an exemplary embodiment of the invention, said plate body fits in a cylinder having a diameter of 30 mm.

In an exemplary embodiment of the invention, said plate body is adapted to fit through a tissue incision of substantially same dimensions as a width of said plate, said width being defined in a direction perpendicular to a long axis of said plate and perpendicular to an insertion axis of said lag screw.

In an exemplary embodiment of the invention, said body is formed of titanium.

In an exemplary embodiment of the invention, said screw is formed of titanium.

In an exemplary embodiment of the invention, said body is formed of a polymer.

In an exemplary embodiment of the invention, said plate body defines at least one hole for fixation of said plate to cortical bone, using a connector.

There is also provided in accordance with an exemplary embodiment of the invention, apparatus for bone implant removal, comprising:

a guide tube;

an implant engaging rod in said tube; and

a hydraulic piston adapted to selectively pull back said rod. Optionally, said apparatus is designed for an expandable implant. Optionally, said guide tube is adapted to collapse said implant.

In an exemplary embodiment of the invention, said rod is adapted to engage by a threading. Alternatively or additionally, said rod is adapted to release a valve on a specific implant, when engaging said implant.

There is also provided in accordance with an exemplary embodiment of the invention, a method of implanting a hip plate, comprising:

inserting a hip plate body to lie against a bone;

assembling a barrel guide in said plate in said body; and

rotationally and axially locking said barrel guide to said plate body. Optionally, said barrel locks upon assembly.

In an exemplary embodiment of the invention, the method comprises attaching a drill guide to said plate body.

In an exemplary embodiment of the invention, the method comprises inserting a lag screw through said barrel guide.

In an exemplary embodiment of the invention, the method comprises limiting axial motion of said lag screw.

In an exemplary embodiment of the invention, the method comprises comprising inserting a hip pin through said plate body.

There is also provided in accordance with an exemplary embodiment of the invention, a method of hip lag screw removal, comprising:

engaging a lag screw using an engaging rod;

pulling back the engaging rod so that the lag screw enters a guide tube. Optionally, said pulling back comprises radially compressing at least a portion of said lag screw. Alternatively or additionally, said engaging comprises releasing an internal pressure in said lag screw. Alternatively or additionally, said pulling back comprises pulling back using hydraulic force.

### BRIEF DESCRIPTION OF THE FIGURES

Some exemplary embodiments of the invention will be further described with reference to the accompanied drawings, in which same number designations are maintained throughout the figures for corresponding and same element and in which:

FIGS. 1A and 1B are perspective views of a plate body, plate barrel and hip peg, assembled in accordance with an exemplary embodiment of the present invention;

FIGS. 2A and 2B are back and crossed-sectional side views, respectively, of a plate and barrel of FIG. 1;

FIG. 2C is an enlarged detail of FIG. 2B, showing a barrel locking mechanism, in accordance with an exemplary embodiment of the invention;

FIGS. 3A and 3B are perspective views of plate body and barrel, according to an alternative exemplary embodiment of the invention for locking the two parts, in which plate body and barrel are connected and locked by a spiral thread and a clamping snap;

FIGS. 4A, 4B and 4C are perspective, top and side views, respectively, of the hip peg of FIG. 1, in accordance with an exemplary embodiment of the present invention;

FIGS. 5A and 5B are perspective and side views, respectively, of a hip pin, in accordance with an exemplary embodiment of the present invention;

FIG. 6 is side view of a cortical screw, in accordance with an exemplary embodiment of the present invention;

FIG. 7 is a perspective view of an aiming device, connected to the plate of FIG. 1, in accordance with an exemplary embodiment of the invention; and

FIG. 8 is a cross-sectional side view of hip peg removal device, connected to the hip peg of FIG. 4, in accordance with an exemplary embodiment of the invention.

### DETAILED DESCRIPTION OF EXEMPLARY EMBODIMENTS

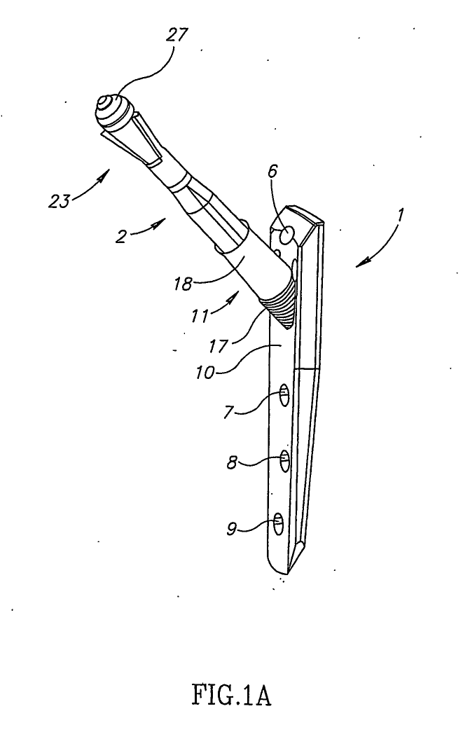

FIGS. 1A and 1B illustrate a hip plate 1 and a hip peg 2, assembled with an optional plate barrel 11, in accordance with an exemplary embodiment of the invention. Optional hip pin(s) and cortical screws(s) are not shown in FIGS. 1A and 1B, for clarity. Plate 1 comprises a plate body 10, generally vertically placed at the external lateral cortex of the femoral bone, into which a plate barrel 11 is inserted. Optionally, barrel 11 serves as a guide hip peg 2 and/or for preventing movement of peg 2 other than along its axis. In an exemplary embodiment of the invention, plate body 10 is a solid component. Alternatively, a jointed plate or a plate assembled from parts may be provided. In an exemplary embodiment of the invention, plate body 10 includes an optionally angled hole 5, for mounting of barrel 11 and/or insertion of hip peg 2. Optionally, provision for a second hip peg is provided, for example, by an optionally angled hole 6, which may be angled at a different angle. Optionally, hole 6 does not use a barrel and/or is used for a smaller diameter hip peg 3 or hip pin. In an exemplary embodiment of the invention, one or more distal holes, for example three holes 7-9, are provided for screws for attachment to the femur (for example as described below). Other attachment methods may be used as well.

In an exemplary embodiment of the invention, holes 5 and 6 are parallel to each other and at an angle 13, for example an angle of about 125° or 135° to 145°, relative to plate body 10. In an exemplary embodiment of the invention, one or more of holes 7-9 intended for screw insertion, are angled to the vertical plane, for example at same or different angles. In an exemplary embodiment of the invention, plate body 10 is shaped to comply with the bone lateral cortex curvature.

Optionally, plate 10 includes a relatively sharp distal end 16 to facilitate tissue dissection.

In an exemplary embodiment of the invention, the length of plate body 10 is between 100-170 mm, with width of, for example, 19 mm. These sizes may vary, for example, for different sized patients and/or different conditions of the femur. In an exemplary embodiment of the invention, barrel 11 is shaped like a cylinder 17 of, for example, 11.5 mm outer diameter, and an internal oval-shaped tube 18 of, for example, 8×10 mm. It is noted, that said dimensions are typical, and may vary and/or a value within the range of dimensions used.

In an exemplary embodiment of the invention, means are provided to prevent rotation of barrel 11. In one example, barrel 11 (and hole 5) are oval, or include another interlocking shape, such as matching protrusions. Optionally, if rotation is prevented, barrel 11 is inserted axially and a selective axial motion prevention lock is provided, for example as described below for a threaded embodiment.

In an exemplary embodiment of the invention, means are provided to prevent rotation of hip peg 2. In one example, the hip peg is oval (e.g., matching an inner diameter shape of at least part of barrel 11). In another example, a set of matching one or more protrusion and one or more recesses or other interlocking design is provided.

It should be noted that, in some embodiments of the invention, axial motion of hip peg 2 relative to plate 10 is possible, typically by motion relative to barrel 11. Optionally, however, barrel 11 does not move axially. In an alternative embodiment, barrel 11 is adapted to move axially.

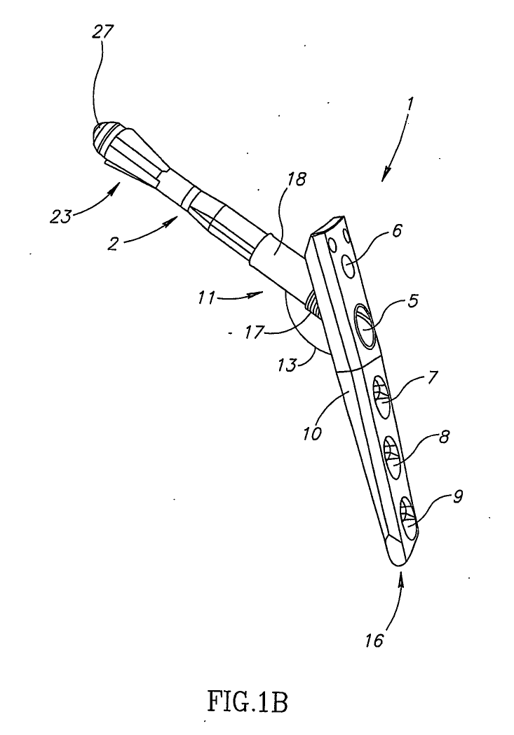

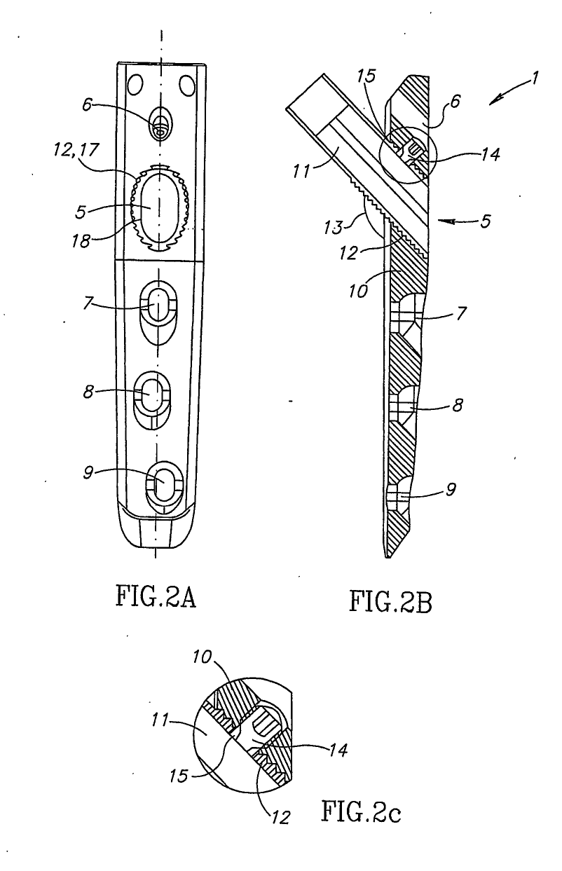

FIGS. 2A and 2B illustrate a back and crossed-sectional side views, respectively, of plate 1. In the example shown, plate body 10 and plate barrel 11 are connected to each other via a thread 12, at an angle 13 of, for example, 135°. Once connected, plate body 10 and barrel 11 are optionally locked to each other to prevent relative motion between them, using a bolt 14 that interlocks with a slot 15 in barrel 11. Optionally, bolt 14 is tightened using a screwdriver, for example using hole 6 for access. Optionally, bolt 14 does not obstruct hole 6, after tightening. FIG. 2C is an enlargement showing bolt 14. In an exemplary embodiment of the invention, a hex-tipped designated screwdriver is provided with a kit including plate 1 and other components, for example as described below.

In an alternative embodiment of the invention, bolt 14 is spring-loaded to lock barell 11. Insertion of barrel 11 pushes away bolt 14 until slot 15 is opposite bolt 14 and bolt 14 can enterslot 15.

Optionally, slot 15 is a through slot so that bolt 14 can interlock with hip peg 2, for example, to control axial motion thereof.

Following is a general description of a minimally invasive surgical technique which may be used to implant the hip plate-screw device, in accordance with some exemplary is embodiments of the invention. Alternatively, an open surgical approach may be used.

(a) The patient is properly prepared and positioned, and all other preoperational procedures are effectuated. For example, the patient may be laid on his side and the incision area sterilized.

(b) A plate size is selected, for example, base don x-ray images, an estimated strength of the bone and/or other information. Multiple plate sizes may be available, for example, 2-4 different sizes.

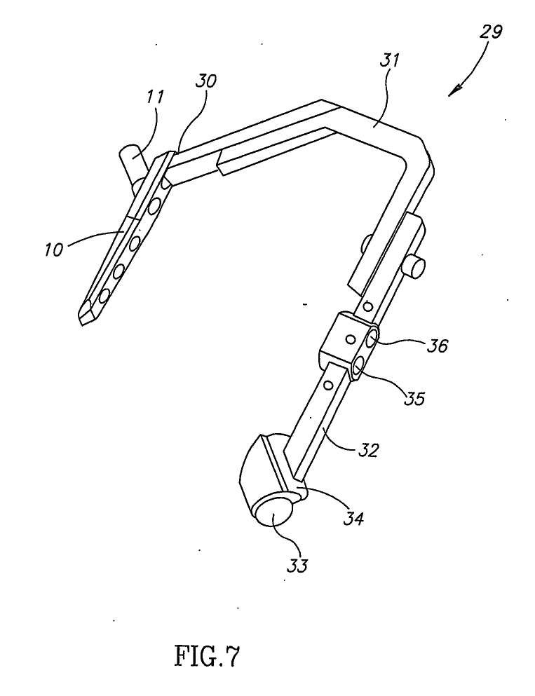

(c) Plate body 10 is optionally connected at a proximal end 30 thereof (or at another portion thereof) to a designated aiming device 29 (described below, FIG. 7).

(d) A small incision, for example between 10 and 30 mm in length (or longer, for example, between 300 and 500 mm in length) is made in the hip.

(e) Using aiming device 29, plate body 10 is introduced through the small incision of soft tissue at the lateral trochanteric area, until it is parallel to the femoral cortex and contacts it. Sharp edge 16 optionally serves to dissect tissue. The aiming device is optionally used to assure correct placement and/or aiming during placement.

(f) A K-wire is optionally introduced into the neck and femoral head via hole 5 in plate body 10 intended for hip peg 2 insertion, and the fracture is optionally reduced.

(g) Barrel 11 is optionally inserted through a second small incision at the thigh, using aiming device 29 and a designated barrel insertion handle. Alternatively, the same incision as used in (d) for the plate body is used for the barrel. Optionally, an opening for the barrel is drilled in the cortical bone.

(h) Barrel 11 is screwed into (or otherwise attached to) its designated threaded hole 12 in plate body 10.

(i) Barrel 11 is optionally locked to plate body 10 by screwing locking bolt 14 of plate body 10, optionally using the designated locking driver. Alternatively, a snap-lock mechanism, for example a spring loaded bolt, or a cantilevered bar, are used.

(j) A stabilizing screw is optionally inserted into distal screw hole 9 of plate body 10.

(k) The K-wire is removed and hip peg 2 is introduced through barrel 11, optionally using hip peg insertion handle and aiming device 29. Optionally, a bore is drilled in the bone first, using barrel 11 as a guide.

(l) A distal section 23 of hip peg 2 is optionally expanded by introducing pressurized sterile saline, optionally utilizing a pump provided with the kit. Other expansion mechanisms, if desired, may be used, for example, air pressure based or mechanical distortion based.

(m) One or more cortical screws 4 (described below) are optionally inserted into distal holes 7-9 of plate body 10 in order to lock plate 1 to the femur (optionally, the stabilizing screw is removed). Other locking means, such as clips and adhesive, may be used.

(n) Optionally, if desired, the axial sliding range of hip peg 2 is limited, by further screwing bolt 14 in plate body 10. In other embodiments, blot 14 springs into a position where it limits motion.

(o) Optionally, a hip pin 3 is inserted through proximal hole 6 of plate body 10, above hip peg 2. Hip pin 3 maybe also an expanding peg.

(p) The incision is closed, for example using methods known in the art.

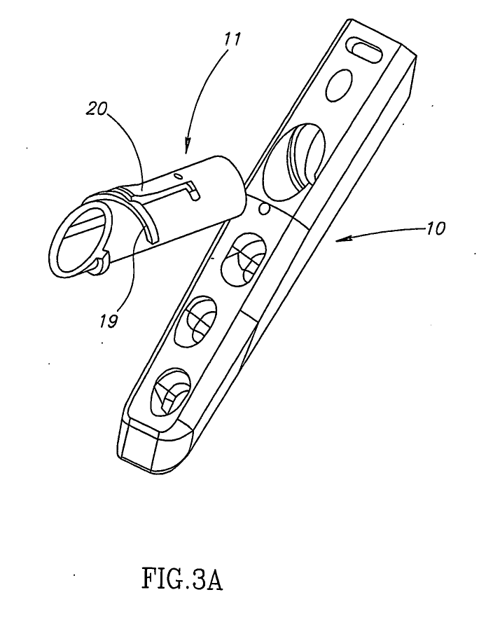

FIGS. 3A and 3B illustrate another exemplary embodiment of the invention, in which plate barrel 11 and plate body 10 are connected by a spiral thread 19. In this exemplary embodiment, locking of plate barrel 11 to plate body 10 is achieved automatically at the end of the rotational connection of plate barrel 11, by a clamping snap 20 in plate barrel 11 fitting into a designated slot 21 in plate body 10. Optionally, no threading is used. Instead, snap locking in both radial and axial directions is provided.

Optionally, multiple threads 19 are provided, for example as two spirals with separate starting points. Optionally, this allows a greater strain to be handled by the threading.

In an exemplary embodiment of the invention, snap 20 and slot 21 are configured to operate even in the presence of bone chips, for example, by snap 20 being configured to push bone chips out along slot 21 and/or into the hollow of barrel 11. Optionally, snap 20 is stiff enough and/or elastically urged with enough force to crush bone chips.

Optionally, the threading is made thick and matches a suitable inner threading on plate 10, so that bone chips cannot clog the threading and prevent assembly.

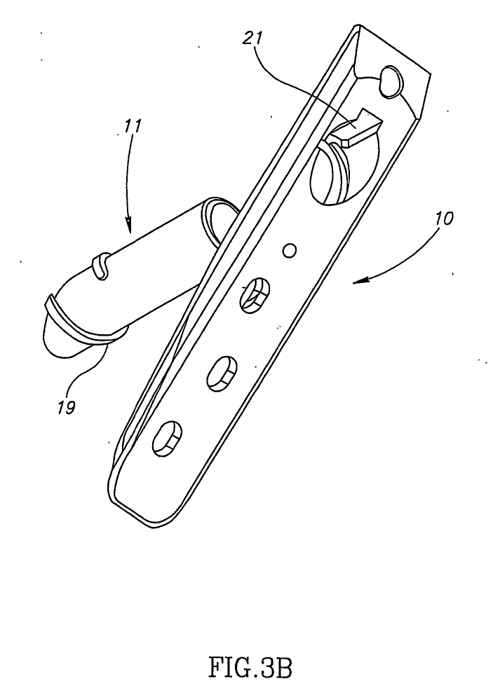

FIGS. 4A, 4B and 4C illustrate perspective, top and side views, respectively, of hip peg 2, in accordance with some exemplary embodiments of the present invention. In an exemplary embodiment of the invention, hip peg 2 comprises an oval shaft 22, with a larger diameter in the vertical plane, and an expandable distal section 23. Oval shaft 22 may have length of, for example, 80, 90, 100, 110 or 120 mm, and diameter of, for example, 8×10 mm. circular cross-sections may be provided as well, in other embodiments. In an exemplary embodiment of the invention, expandable distal section 23 is constructed of a thin-wall membrane 23A with, for example, three longitudinal bars 24-26. In an exemplary embodiment of the invention, distal section 23 is folded to create a reduced-diameter configuration. Upon expansion, the material of hip peg distal section 23 is unfolded, to gain its expanded configuration with bars 24-26, that enhance the abutment of hip peg 2 within the femoral head. A smaller or greater number of bars may be used. Optionally, other designs are used, for example round protrusions.

In an exemplary embodiment of the invention, at least part of expandable portion 23 of hip peg 2 is annealed or stress relieved, depending on material type, for example by heat treatment, to allow it to deform to a greater extent without cracking.

In an exemplary embodiment of the invention, expandable distal section 23 of hip peg 2 may end with a conical shaped distal end 27. Various designs may be selected, for example depending on whether hip peg 2 is inserted with or without drilling.

In an exemplary embodiment of the invention, the diameter of distal section 23 increases upon expansion from, for example, 8 mm (pre-expansion reduced diameter) up to, for example, 12 mm, depending on the quality of the bone. Other exemplary expansion ratios include 1:1.4, 1:2, 1:2.5 and 1:3. In some cases, expansion of distal section 23 of hip peg 2 within the femoral head increases bone density surrounding the expanded section of the implant, and thus improves implant grip within the bone. Expansion is optionally achieved by introducing liquid, for example sterile saline, under pressure, into distal section 23 of hip peg 2, in accordance with an exemplary embodiment of the invention. An external manual pump, for example, may be used for provision of pressurized liquid. In an exemplary embodiment of the invention, hip peg 2 may contain a one-way valve to keep pressurized liquid inside expanded section 23 (not shown in FIG. 4). Alternatively, pressurized liquid does not remain inside hip peg.

In an exemplary embodiment of the invention, shaft 22 of hip peg 2 contains a passage for provision of fluid into expandable distal section 23. Alternatively or additionally, shaft 22 includes a channel for a guide wire or guide tool.

In an exemplary embodiment of the invention, movement (sliding) of hip peg 2 within plate barrel 11 can be limited, if desired and required, The limited sliding potential is intended to prevent migration of hip peg 2 in both directions and damaging, for example, the acetabulum. Sliding limitation may be performed by rotating a bolt 14 in plate body 10, the bolt optionally accessible from the proximal end, for example with the designated screwdriver, until bolt 14 inserts into a matching slot 28 in hip peg 2. If said slot 28 has, for example, a length of 20 mm, and bolt 14 has, for example, a length of 1 mm, sliding ability of hip peg 2 can be limited to 19 mm. Alternatively, sliding potential of hip peg 2 may be restricted, for example, to 15 mm, 18 mm or 20 mm. Other degrees of restriction may be provided, for example, depending on the patient.

Optionally, slot 28 is spiral, so that peg 2 can move axially and rotate. Optionally, peg 2 is slightly curved.

In an exemplary embodiment of the invention, a same bolt 14 in plate body 10 is used for limiting hip peg 2 and for locking barrel 11 to plate body 10. Alternatively, separate mechanisms are used for said two actions, for example two separately controllable or spring-loaded bolts.

In an exemplary embodiment of the invention, the locking mechanism of hip peg 2 may be incorporated into other implantable systems, for example a proximal femoral intramedullary nailing system that includes a hip peg component which is inserted into the femoral head, through a designated hole in the intramedullary nail. For example, a snap-type axial locking may be provided. Optionally, a barrel is provided as well.

Optionally, a back end of barrel 11 is covered by a cap which includes a spring or shock absorber to cushion axial motion of peg 2. Optionally, the cap is attached by threading, optionally in an opposite direction from barrel 1 1.

Optionally, a bolt 14 is provided for axially locking peg 2. Optionally, increased advancing of bolt 14 will prevent any axial motion. Optionally, slot 28 of peg 2 has a non-uniform depth, for example a stepped or an inclined depth, so that different bolt positions translate into different degrees of axial motion. Optionally, an aperture is provide din barrel 11 to allow passage of bolt 14 to peg 2.

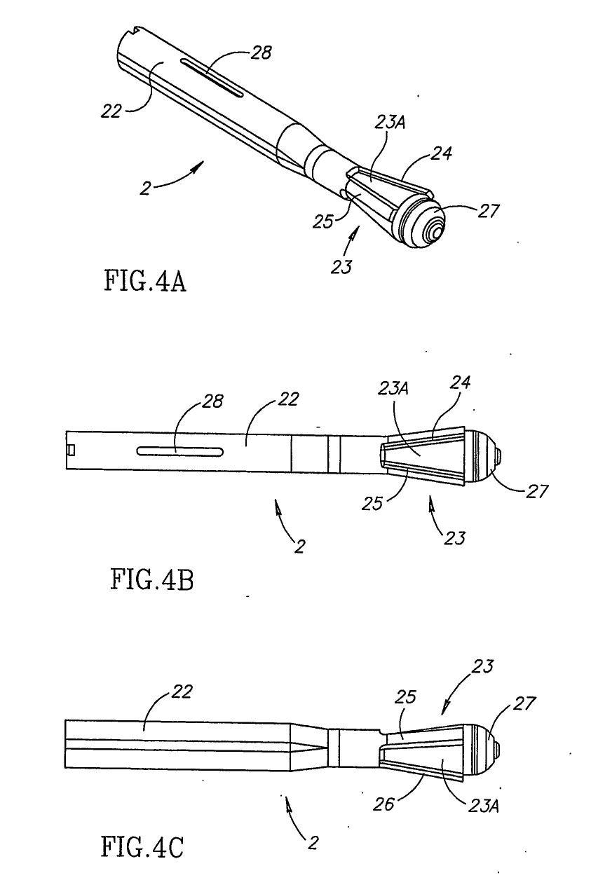

FIGS. 5A and 5B illustrate a hip pin 3, in accordance with an embodiment of the invention. Hip pin 3 is optionally used to provide additional fixation of a bone fragment, if needed, and for stabilizing the bone in case of torsional instability (e.g., caused by rotation of the trochanter relative to peg 2). When used, hip pin 3 is inserted into proximal, angled hole 6 in plate body 10. In an exemplary embodiment of the invention, hip pin 3 diameter may be, for example, 5 mm, and its length may be, for example, 60 mm, 70 mm, 80 mm, 90 mm or 100 mm. Various other sizes can be used, depending, for example, on the patient.

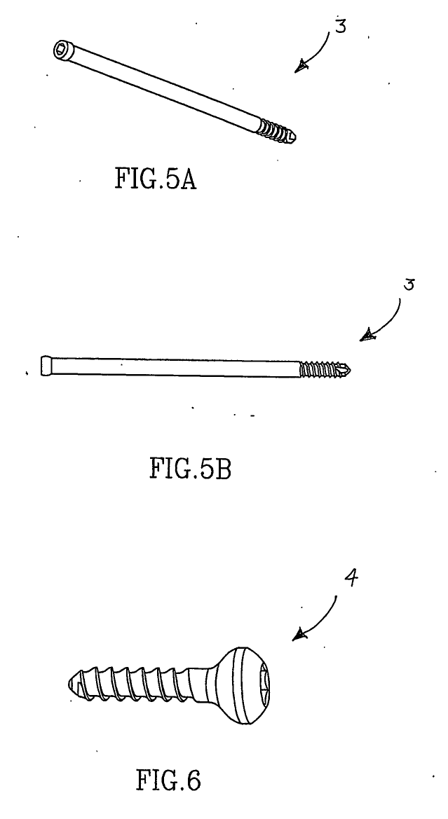

FIG. 6 illustrates a cortical screw 4, in accordance with some embodiments of the invention. Screws 4 are inserted into distal holes 7-9 of plate body 10, for attaching the distal section of plate 1 to the cortical bone. The number of screws 4 used may be dependent on the length of plate body 10. In an exemplary embodiment of the invention, the maximum number of screws is three. Alternatively, the maximal number may be, for example, four, five, six or seven. In an exemplary embodiment of the invention, screws may have a diameter of, for example, 4.5 mm, and a length of, for example, 25 mm, 30 mm, 35 mm, 40 mm, 45 mm, 50 mm, 55 mm or 60 mm. Various other sizes can be used, depending, for example, on the patient.

Optionally, plate 1, hip peg 2, hip pin 3 and screws 4 are made of titanium. Alternatively or additionally, these components may be constructed from other materials, for example 316 stainless steel or other metal or polymer. Optionally, biocompatible materials with biomechanical properties similar to those of titanium, are selected. In an exemplary embodiment of the invention, different device components are constructed from different materials.

It is noted that all the above-mentioned components are not restricted to the above-mentioned dimensions. For example, dimensions are typical, and may vary, for example with the exemplary numbers being ranges.

Designated instruments are optionally provided to assist performing minimally invasive implantation of said hip plate-screw device, in accordance with an exemplary embodiment of the invention. Instrumentation may include, for example and among other, a plate insertion handle (aiming device) 29, hip peg insertion handle, barrel insertion handle, guide and drill sleeves, triple reamer, pump, stabilizing screw, locking driver, hip peg removal device 37, screwdriver, k-wires and drill bits. In an exemplary embodiment of the invention, instruments may be provided in one or more kits. In an exemplary embodiment of the invention, instruments may be provided sterile. Alternatively, instruments are provided non-sterile, to be sterilized by the user prior to their use.

FIG. 7 illustrates the aiming device 29 (connected to a plate body 10), in accordance with an exemplary embodiment of the invention. Aiming device 29 comprises a curved handle 31 and a guide sleeve handle 32, incorporating holes 33-36 for insertion of guide sleeves and drill sleeves to assist in drilling the required holes for hip peg 2, hip pin 3 (if needed), and/or screws 4. Aiming device 29 is connected to proximal end 30 of plate body 10, for example by screws, to provide stable connection and to prevent relative motion between plate body 10 and aiming device 29. In an exemplary embodiment of the invention, the various holes are used to guide a drilling bore direction. In some embodiments, a bore for barrel 11 is formed before placing plate body 11. In some embodiments, the bore is formed through plate body 11. Optionally, bolt 14 is tightened after aiming device 29 is removed. Alternatively, an aperture (not shown) is provided in aiming device 29, for access to bolt 14.

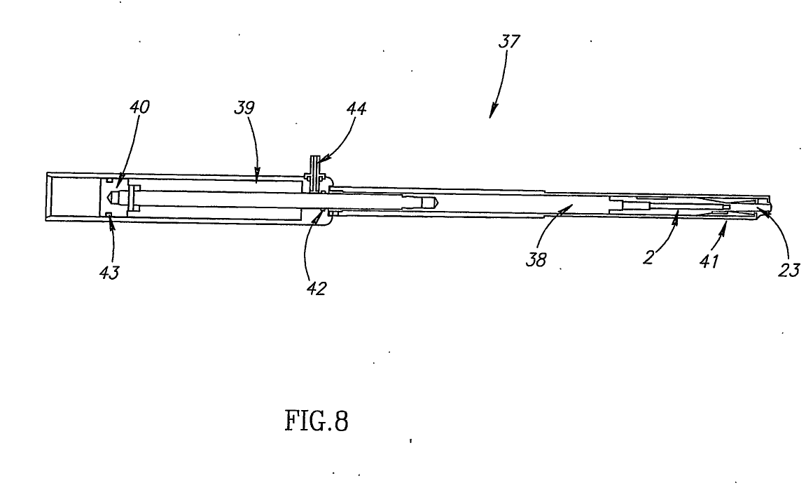

FIG. 8 illustrates a hydraulic removal device 37 for extraction of hip peg 2, if required, in accordance with some exemplary embodiments of the invention. Hip peg removal device 37 comprises a rod 38 that is connected to hip peg 2; a cylinder 39, containing a piston 40 that is connected to rod 38; and a tube 41 into which hip peg 2 is pulled.

In an exemplary embodiment of the invention, hip peg 2 and rod 38 are connected to each other by a threading. In an exemplary embodiment of the invention, cylinder 39 may be sealed with, for example, one or more o-rings 42-43. Optionally, two o-rings are used. In an exemplary embodiment of the invention, tube 41 may have a smaller diameter than the diameter of expanded section 23 of hip peg 2.

In an exemplary embodiment of the invention, in order to extract hip peg 2 from patient body hip peg removal device 37 may be connected via a quick connector 44 to a manual pump, which delivers pressurized liquid, for example sterile saline, into cylinder chamber 39. Once sufficient pressure is built in chamber 39, piston 40 moves backwards and exerts axial force to extract (pull) rod 38 and hip peg 2 into said tube 41. Other mechanism can be used for pulling back, for example, a knob that turns a screw thereby pulling back piston 40. Optionally, a high mechanical gain is provided, to avoid the user from applying high forces which can cause inadvertent movement.

In an exemplary embodiment of the invention, the diameter of expanded distal section 23 of hip peg 2 may be reduced during extraction, by mechanical forces exerted on hip peg 2 while hip peg 2 is pulled through tube 41 that has a smaller diameter than the diameter of an expanded hip peg portion 23. Alternatively or additionally, reduction in diameter of expanded hip peg portion 23 may be achieved by piercing a one-way valve in hip peg 2, intended to maintain pressurized liquid inside expanded portion 23, to allow pressure decrease and easier removal. Piercing may be performed, for example, while connecting hip peg 2 to removal device rod 38, by a sharp extension of rod 38. In an exemplary embodiment of the invention, the one-way valve of hip peg 2 is positioned so that threading rod 38 into peg 2 pushes a distal end of rod 38 against the valve and releases the pressure in hip peg 2.

In an exemplary embodiment of the invention, removal device 37 is not limited to extraction of hip peg 2 component of hip plate-screw system, but is suitable for extraction of other types of implanted hip pegs, such as hip peg component of intramedullary nailing system. Alternatively or additionally, the removal device may be used to extract an intramedullary nail from the bone.

In an exemplary embodiment of the invention, barrel 11 is removed by opening bolt 14 (e.g., using the screw driver or using a tool that pushes snap 20 out of slot 21 0. In an exemplary embodiment of the invention, the tool comprises a tube with a projecting tooth that is oriented outwards and backwards. In use, the tube is advanced past snap 20 and retracted so that tooth engages snap 20 and allows barrel 11 to be rotated and/or otherwise removed.

In another embodiment of the invention, where the implant is proximally or distally threaded, for example to a plate, the piston also rotates while moving backwards, enabling rotation of implant during extraction. In an exemplary embodiment of the invention, the piston rod is hollow and a spiral is located inside it. Rotational movement of piston is simultaneously achieved with its backwards movement. In an alternative embodiment, piston 40 has a threading matching a protrusion or threading in chamber 39.

Various features of devices and methods have been described. It should be appreciated that combinations of the above features are also considered to be within the scope of some exemplary embodiments of the invention. It should also be appreciated that some of the embodiments are described only as methods or only as apparatus, however the scope of the invention includes both methods for using apparatus and apparatus for applying methods. The scope of the invention also covers machines for creating the apparatus described herein. In addition, the scope of the invention also includes methods of using, constructing, calibrating and/or maintaining the apparatus described herein. When used in the following claims or in the text above, the terms “comprises”, “comprising”, “includes”, “including” or the like mean “including but not limited”.

1. A hip plate, comprising:
a plate body adapted to be attached to a bone; and
at least one lag screw adapted for insertion through the plate and into a bone section that is offset from said bone and rotationally lockable to said plate.

2. A plate according to claim 1, wherein said bone is a femur and wherein said offset is a femoral head.

3. A plate according to claim 1, comprising a barrel guide having an inner diameter adapted to contain a shaft of said lag screw and axially guide a motion of said lag screw.

4. A plate according to claim 3, wherein said barrel is rotationally locked to said plate and wherein said screw is rotationally locked to said barrel.

5. A plate according to claim 3, wherein said barrel guide is adapted for attachment to said plate after said plate is implanted in a human body.

6. A plate according to claim 3, wherein said barrel is attached to said plate using a threading.

7. A plate according to claim 3, wherein said barrel is axially locked to said plate preventing motion of said barrel along a main axis thereof.

8. A plate according to claim 7, wherein said barrel is locked using a manually positioned locking element.

9. A plate according to claim 7, wherein said barrel is locked using a self-engaging element.

10. A plate according to claim 1, wherein said lag screw has an expandable distal end.

11. A plate according to claim 10, wherein said distal end is inflatable.

12. A plate according to claim 10, wherein said distal end comprises a plurality of protrusions.

13. A plate according to claim 12, wherein said protrusions comprise axial bars.

14. A plate according to claim 10, wherein said expandable end is treated to increase elongation.

15. A plate according to claim 1, wherein said lag screw comprises a one way fluid valve.

16. A plate according to claim 15, wherein said valve is adapted to release said fluid when said valve is axially depressed towards said a distal end of said lag screw.

17. A plate according to claim 1, wherein said lag screw includes an axial motion limiter.

18. A plate according to claim 17, wherein said limiter comprises a slot adapted to be engaged by a matching protrusion.

19. A plate according to claim 17, wherein said limiter allows some axial motion.

20. A plate according to claim 1, wherein a shaft section of said peg has a cross-section that is not circular.

21. A plate according to claim 20, wherein said peg has an oval cross-section.

22. A plate according to claim 1, wherein an end of said plate is sharp enough to push away tissue.

23. A plate according to claim 1, wherein said plate body fits in a cylinder having a diameter of 30 mm.

24. A plate according to claim 1, wherein said plate body is adapted to fit through a tissue incision of substantially same dimensions as a width of said plate, said width being defined in a direction perpendicular to a long axis of said plate and perpendicular to an insertion axis of said lag screw.

25. A plate according to claim 1, wherein said body is formed of titanium.

26. A plate according to claim 1, wherein said body is formed of a polymer.

27. A plate according to claim 1, wherein said screw is formed of titanium.

28. A plate according to claim 1, wherein said plate body defines at least one hole for fixation of said plate to cortical bone, using a connector.

29. Apparatus for bone implant removal, comprising:
a guide tube;
an implant engaging rod in said tube; and
a hydraulic piston adapted to selectively pull back said rod.

30. Apparatus according to claim 29, wherein said apparatus is designed for an expandable implant.

31. Apparatus according to claim 30, wherein said guide tube is adapted to collapse said implant.

32. Apparatus according to claim 30, wherein said rod is adapted to engage by a threading.

33. Apparatus according to claim 30, wherein said rod is adapted to release a valve on a specific implant, when engaging said implant.

34. A method of implanting a hip plate, comprising:
inserting a hip plate body to lie against a bone;
assembling a barrel guide in said plate in said body; and
rotationally and axially locking said barrel guide to said plate body.

35. A method according to claim 34, wherein said barrel locks upon assembly.

36. A method according to claim 34, comprising attaching a drill guide to said plate body.

37. A method according to any of claim 34, comprising inserting a lag screw through said barrel guide.

38. A method according to claim 34, comprising limiting axial motion of said lag screw.

39. A method according to claim 34, comprising inserting a hip pin through said plate body.

40. A method of hip lag screw removal, comprising:
engaging a lag screw using an engaging rod;
pulling back the engaging rod so that the lag screw enters a guide tube.

41. A method according to claim 40, wherein said pulling back comprises radially compressing at least a portion of said lag screw.

42. A method according to claim 40, wherein said engaging comprises releasing an internal pressure in said lag screw.

43. A method according to claim 40, wherein said pulling back comprises pulling back using hydraulic force.
